(19) United States
(12) Patent Application Publication
(10) Pub. No.: US 2003/0190351 A1
(43) Pub. Date: Oct. 9, 2003

(54) Extended release venlafaxine formulations

(75) Inventors: Johannes Jan Platteeuw, s'Hertogenbosch (NL); Inocencia Margallo Lana, Barcelona (ES); Arturo Siles Ortega, Barcelona (ES)

Correspondence Address: FLESHNER & KIM, LLP, P.O. Box 221200, Chantilly, VA, 20153-1200, US

(73) Assignee: Synthon BV

(21) Appl. No.: 10/397,350

(22) Filed: Mar. 27, 2003

Related U.S. Application Data
(60) Provisional application No. 60/367,735, filed on Mar. 28, 2002.

(51) Int. Cl. A61K 31/137; A61K 9/20

(52) U.S. Cl. 424/465; 514/649

(57) ABSTRACT

A pharmaceutical composition is provided comprising venlafaxine hydrochloride and sodium carboxymethyl cellulose. The composition can be formulated to provide controlled release of the venlafaxine hydrochloride.

This application claims the benefit of priority under 35 U.S.C. §119 from prior U.S. provisional application serial No. 60/367,735, filed Mar. 28, 2002, the entire contents of which are incorporated herein by reference.

### BACKGROUND OF THE INVENTION

The present invention relates to extended release formulations of venlafaxine hydrochloride and to the use thereof in treating various diseases or conditions.

Venlafaxine is the common name for the compound 1-[2-(dimethylamino)-1-(4-methoxyphenyl) ethyl]cyclohexanol, having the structure shown below.

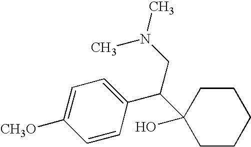

U.S. Pat. No. 4,535,186 describes a class of hydroxycycloalkanephenethyl amines as being useful antidepressants and exemplifies the compound now known as venlafaxine hydrochloride as one of the suitable species. Venlafaxine hydrochloride is approved for sale in various countries including the United States of America under the brand name EFFEXOR® (Wyeth Ayerst). It is available as an immediate release tablet and as an extended release capsule under the brand name EFFEXOR® (Wyeth Ayerst) and EFFEXOR XR® (Wyeth Ayerst), respectively.

Venlafaxine has been the subject of various research endeavors. For example, U.S. Pat. No. 5,043,466 describes a process for making cyclohexanol derivatives in a specified solvent composition. Example 3 of this patent shows the synthesis of venlafaxine as the hydrochloride salt thereof.

U.S. Pat. No. 6,274,171 and related EP 0 797 991A1 disclose encapsulated extended release formulations for venlafaxine hydrochloride. These patents indicate that commercial venlafaxine hydrochloride tablets were administered two or three times daily, but that due to variations in the drug concentration in the patient's blood plasma caused by such a dosing regimen, unwanted side effects, especially nausea and vomiting were common. A once daily, encapsulated extended release dosage form is disclosed that provides a flattened drug plasma profile and reduces these side effects. The encapsulated dosage form is taught to comprise spheroids of venlafaxine hydrochloride, microcrystalline cellulose, and hydroxypropylmethylcellulose (HPMC). These spheroids are coated with a mixture of ethyl cellulose and HPMC. By providing an appropriate amount of the coating, the desired blood plasma profile can be obtained. An acceptable batch of coated spheroids will meet the following in vitro dissolution profile:

| | Average % venlafaxine |
|---|---|
| Time (hours) | hydrochloride released |
| 2 | <30 |
| 4 | 30-55 |
| 8 | 55-80 |
| 12 | 65-90 |
| 24 | >80 |

using USP Apparatus 1 (basket) at 100 rpm in purified water at 37° C. The coated spheroids can be from a single batch or represent a blend of different batches.

U.S. Pat. No. 6,274,171 and EP 0 797 991 also state that forming an extended release dosage from of venlafaxine hydrochloride was difficult in part due to the high water solubility of the hydrochloride salt. In fact, these patents disclose that “[n]umerous attempts to produce extended release tablets by hydrogel technology proved to be fruitless because the compressed tablets were either physically unstable (poor compressibility or capping problems) or dissolved too rapidly in dissolution studies.” See U.S. Pat. No. '171 at column 4, lines 60-65 and EP '991 at page 3 lines 35-37. Unlike the encapsulated extended release formulations described in these patents, a hydrogel extended release venlafaxine hydrochloride tablet is taught to typically exhibit a dissolution profile wherein 40%-50% is released at 2 hours, 60%-70% is released at 4 hours, and 85%-100% is released at 8 hours.

WO99/22724 also discloses encapsulated venlafaxine hydrochloride extended release dosage forms. These formulations differ from those in U.S. Pat. No. '171 and EP '991 in that the spheroid is substantially free of HPMC. Apparently HPMC can be omitted from the spheroid when smaller amounts of venlafaxine hydrochloride are employed.

Although a venlafaxine extended release capsule has been produced, it would be advantageous to provide a less complicated dosage form that nonetheless provides extended release of venlafaxine.

### SUMMARY OF THE INVENTION

The present invention relates to the surprising discovery that sodium carboxymethyl cellulose can provide superior controlled release of venlafaxine hydrochloride. Accordingly, a first aspect of the present invention relates to a pharmaceutical composition that comprises venlafaxine hydrochloride and sodium carboxymethyl cellulose. The pharmaceutical composition includes finished dosage forms as well as intermediates/precursors thereof such as tabletting blends. The pharmaceutical compositions are generally in tablet form and can exhibit good extended release properties such that a twice daily or more preferably a once daily dosage form is provided.

Another aspect of the present invention relates to a method for treating venlafaxine-treatable diseases and conditions, especially depression, by administering an effective amount of the above-described pharmaceutical composition to a patient in need thereof. The pharmaceutical composition is preferably administered once daily in the form of one or two tablets.

### DETAILED DESCRIPTION OF THE INVENTION

The present invention is based on the discovery of a hydrogel polymer that can adequately control the release of venlafaxine hydrochloride. Contrary to the investigations reported in the prior art, a hydrogel technology has been found that can extend the release of the very water soluble venlafaxine hydrochloride. Specifically, the polymer sodium carboxymethyl cellulose can be used to formulate a venlafaxine hydrochloride hydrogel tablet that can exhibit a more desirable release profile.

Sodium carboxymethyl cellulose useable in the present invention is desirably not crosslinked. Further, higher viscosity polymers are preferred over lower viscosity polymer forms. While higher molecular weight is normally associated with higher viscosity, such is not required and between the two physical properties viscosity is more important. Sodium carboxymethyl cellulose is well known in the art and is commercially available.

The venlafaxine hydrochloride used in the present invention can be based on the racemate or mixture of enantiomers of venlafaxine, or, on the pure or substantially pure (+) or (−) enantiomer of venlafaxine (hereinafter referred to as (+)-venlafaxine hydrochloride and (−)-venlafaxine hydrochloride). All of these possibilities are included within the meaning of “venlafaxine hydrochloride,” unless specifically noted otherwise, and can be made by synthetic techniques known in the art.

The pharmaceutical compositions of the present invention contain venlafaxine hydrochloride and sodium carboxymethyl cellulose. As pharmaceutical compositions, the kind and amount of additional ingredients is limited to those excipients that are pharmaceutically acceptable and in pharmaceutically acceptable amounts. Typical excipients are described hereinafter. The pharmaceutical compositions of the present invention generally contain, by weight, 10%-50% of the venlafaxine hydrochloride and 30% to 75%, more typically 30% to 50%, of the sodium carboxymethyl cellulose. In some embodiments it is preferable to have the ratio of venlafaxine hydrochloride to sodium carboxymethyl cellulose within the range of 0.8-1.2:1, more preferably approximately 1:1, respectively. The pharmaceutical compositions generally take the form of a tablet, in particular a hydrogel tablet, but are not limited thereto. The pre-tablet or pre-compression blends are also included as are tablets that are further treated such as by coating, packaging, etc.

A “hydrogel tablet” is one that contains a hydrophilic matrix material that swells or “gels” upon contact with water to thereby slow the diffusion release of the active ingredient. Sodium carboxymethyl cellulose is a hydrophilic matrix material. Other hydrophilic matrix materials that can be combined with sodium carboxymethyl cellulose in forming the hydrogel include other celluloses such as methylcelluloses (i.e. having a viscosity of 400 cP to 4000 cP), hydroxyethylcellulose, and HPMC; polysaccharides such as galactomannanes, potassium alginates, sodium alginates, agar-agar, carrageen, arabic gum, and sterculia gum; polyacrylates such as CARBOPOL 934, EUDRAGIT LD 35; Noveon or polycarbophils; and other water swellable polymers such as polyvinyl alcohol. Generally the sodium carboxymethyl cellulose comprises the majority of the matrix material and typically accounts for at least 50%, more commonly at least 60%, still more commonly at least 65%, and in many embodiments at least 70% or at least 80%. A preferred embodiment comprising a combination of matrix materials includes sodium carboxymethyl cellulose with HPMC and/or microcrystalline cellulose. The amount of sodium carboxymethyl cellulose in these embodiments is generally in the range of 50% to 90%, more commonly 65% to 85%.

Additional pharmaceutically acceptable excipients are well known in the art and include, without limitation, diluents, fillers, binders, lubricants, disintegrants, glidants, colorants, pigments, taste masking agents, sweeteners, and plasticizers. The excipient(s) are selected based in part on the dosage form, the intended mode of administration, the intended release rate, and manufacturing reliability. Typically the composition may further comprise fillers and lubricants in order to assure good properties of the composition and good tabletting. Suitable fillers are, e.g. calcium hydrogenphosphate, microcrystalline cellulose or lactose, suitable lubricants are magnesium stearate or talc.

The hydrogel tablet of the present invention is an extended release dosage form. An extended release dosage form as used herein means that in a dissolution test using USP Apparatus 1 (basket) at 100 rpm in purified water at 37° C., less than 80% of the venlafaxine hydrochloride is dissolved during the first two hours, more typically less than 50%, and preferably less than 30% of the venlafaxine hydrochloride is dissolved during the first two hours. Extended release tablets generally allow for twice a day, or more preferably once a day dosing, to provide 24 hour therapeutic blood plasma levels of venlafaxine to the patient. In this regard, the most preferred dosage form is one which provides once daily dosing. Such a composition should meet the following in vitro dissolution profile:

| | Average % venlafaxine |
|---|---|
| Time (hours) | hydrochloride released |
| 2 | <30 |
| 4 | 30-55 |
| 8 | 55-80 |
| 12 | 65-90 |
| 24 | >80 |

using USP Apparatus 1 (basket) at 100 rpm in purified water at 37° C.

In terms of in vivo performance, the extended release venlafaxine hydrochloride pharmaceutical composition according to the present invention preferably exhibits on average a maximum venlafaxine blood plasma level not earlier than 4 hours, more preferably not earlier than 6 hours after administration of the composition. Typically the average peak plasma level is reached between 4 and 10 hours, more preferably between 6 and 8 hours after administration. In this regard, it is preferred in some embodiments to be bioequivalent to the commercially available EFFEXOR XR®.

The tablets may be produced by any standard tabletting technique, e.g. by wet granulation, dry granulation or direct compression. The tabletting methods that do not employ a solvent (“dry processes”) are generally preferable.

In general, dry granulation procedures comprise mixing the solid excipients (except lubricants), compacting the mixture in a compactor (e.g. a roller compactor), or double compression, milling the compacted mass, screening the milled granules, mixing with a lubricant and compressing the mixture into tablets. Direct compression procedures generally comprise mixing the solid excipients in one or more stages and compressing the uniform mixture into tablets. After tablet formation, the tablets may optionally be coated such as by a film coat. The coating may serve as an environmental barrier to light, heat or moisture, as a taste masking aide, or as an enteric coating. While the later is not common with hydrogel technology, it may be advantageous given the pH sensitivity of the sodium carboxymethyl cellulose, to insure extended release. Alkaline or buffering materials can also be incorporated into the coating, the outer portion of the tablet or throughout the tablet to assist in pH control, if necessary. In one embodiment, a further overcoat layer containing venlafaxine hydrochloride is provided to allow initial release, as desired, in the low pH of the stomach while the remainder, generally the majority (i.e., 90% or more) of the venlafaxine hydrochloride, is isolated under the enteric coating. This outer drug layer can be dissolving or diffusing in nature.

A unit dose, i.e. one tablet, of the pharmaceutical composition of the present invention generally contains from 2 mg to 300 mg, more typically 30 mg to 300 mg of venlafaxine hydrochloride. Contemplated doses include 37.5 mg, 75 mg, 100 mg, 150 mg, 200 mg, and 300 mg strengths. For clarity, all amounts of venlafaxine hydrochloride are expressed in terms of the weight of the free base contained in the hydrochloride salt, as is conventional in the art.

The venlafaxine hydrochloride compound of the present invention can be used to treat any disease or condition that is treatable by venlafaxine. A venlafaxine-treatable disease or condition is one that could be improved by a serotonin or norepinephrine uptake inhibitor and specifically includes, without limitation, depressions, panic disorder, generalized anxiety disorder, obesity, post-traumatic stress disorder, late luteal phase dysphoric disorder, attention deficit disorders, Gilles de la Tourette syndrome, bulimia nervosa, and Shy Drager syndrome. See published U.S. patent application US 2001/0012855 A1 for a description of the uses of venlafaxine and salts thereof. The pharmaceutical composition of the present invention can be used to treat such conditions by administering an effective amount to a patient in need thereof. An effective amount is generally known in the art and/or determined using routine skill. Typically the effective amount for a human contains 30 to 300 mg of venlafaxine per day. The patients used herein include human and non-human mammals such as dogs, cats, and horses. Preferably, the venlafaxine hydrochloride compound is administered orally via one or two unit dosage forms (tablets) of the present invention as described above.

The entire disclosure in each of the patents mentioned in the above description is incorporated herein by reference. The invention will be further described with reference to the following non-limiting examples.

### EXAMPLES

### Example 1

Tablet composition comprising venlafaxine hydrochloride

Composition of a tablet:

| | |
|---|---|
| Venlafaxine hydrochloride | 133.5 mg |
| Sodium carboxymethyl cellulose (Blanose [TM] 7HF) | 133.5 mg |
| Microcrystalline cellulose (Avicel[TM] PH101 ) | 30.0 mg |
| Hydroxypropylmethylcellulose (Methocel[TM] K4M) | 30.0 mg |
| Magnesium stearate | 3.0 mg |

Modus operandi: compaction followed by compression.

Round biconvex tablets of 9 mm diameter and having total mass of 330 mg were made by dry compaction and compression.

Dissolution rate (USP1, water, 37° C.) was determined by UV spectrophotometry and is expressed in % of the declared amount:

| | |
|---|---|
| 2 hrs | 17.8% |
| 4 hrs | 33.2% |
| 8 hrs | 59.4% |
| 12 hrs | 82.2% |
| 24 hrs | 103.1% |

### Example 2

Composition of a tablet:

| | |
|---|---|
| Venlafaxine hydrochloride | 133.5 mg |
| Sodium carboxymethyl cellulose (Blanose [TM] 7HF) | 133.5 mg |
| Microcrystalline cellulose (Avicel[TM] PH101 ) | 30.0 mg |
| Magnesium stearate | 3.0 mg |

Modus operandi: compaction followed by compression.

Round biconvex tablets of 9 mm diameter and having total mass of 300 mg were made by dry compaction and compression.

Dissolution rate (USP1, water, 37° C.) was determined by UV spectrophotometry and is expressed in % of the declared amount:

| | |
|---|---|
| 2 hrs | 23.1% |
| 4 hrs | 44.0% |
| 8 hrs | 78.9% |
| 12 hrs | 100.4% |
| 24 hrs | 104.4% |

The invention having been described, it will be readily apparent to those skilled in the art that further changes and modifications in actual implementation of the concepts and embodiments described herein can easily be made or may be learned by practice of the invention, without departing from the spirit and scope of the invention as defined by the following claims.

We claim:

1. A pharmaceutical composition comprising venlafaxine hydrochloride and sodium carboxymethyl cellulose.

2. The pharmaceutical composition according to claim 1, wherein said venlafaxine hydrochloride is racemic venlafaxine hydrochloride.

3. The pharmaceutical composition according to claim 1, wherein said venlafaxine hydrochloride is (+)-venlafaxine hydrochloride or (−)-venlafaxine hydrochloride.

4. The pharmaceutical composition according to claim 1, wherein said venlafaxine hydrochloride and said sodium carboxymethyl cellulose are present in a weight ratio of 0.8-1.2:1, respectively.

5. The pharmaceutical composition according to claim 4, wherein said weight ratio is about 1:1.

6. The pharmaceutical composition according to claim 1, wherein said composition is in the form of a tablet containing a pharmaceutically effective amount of venlafaxine hydrochloride.

7. The pharmaceutical composition according to claim 6, wherein said tablet contains 10%-50% of said venlafaxine hydrochloride and 30% to 75% of said sodium carboxyrnethyl cellulose.

8. The pharmaceutical composition according to claim 7, wherein said venlafaxine hydrochloride and said sodium carboxymethyl cellulose are present in said tablet in a weight ratio of about 1:1.

9. The pharmaceutical composition according to claim 6, wherein said tablet contains 30 to 300 mg of venlafaxine as said venlafaxine hydrochloride.

10. The pharmaceutical composition according to claim 6, wherein said tablet further comprises hydroxypropyl methylcellulose, microcrystalline cellulose, or a combination thereof.

11. The pharmaceutical composition according to claim 6, which further comprises a coating.

12. The pharmaceutical composition according to claim 6, wherein said tablet has a dissolution profile wherein no more than 30% of said venlafaxine hydrochloride is released during the first two hours in a USP Apparatus 1 (basket) at 100 rpm in purified water at 37° C.

13. The pharmaceutical composition according to claim 12, wherein said tablet dissolution profile meets the following criterion:

| | Average % venlafaxine |
|---|---|
| Time (hours) | hydrochloride released |
| 2 | <30 |
| 4 | 30-55 |
| 8 | 55-80 |
| 12 | 65-90 |
| 24 | >80 |

using USP Apparatus 1 (basket) at 100 rpm in purified water at 37° C.

14. A method for treating a venlafaxine-treatable disease or condition, which comprises administering to a patient in need thereof an effective amount of the pharmaceutical position according to claim 1.

15. The method according to claim 14, wherein said venlafaxine-treatable disease or condition is depression.

16. The method according to claim 15, wherein said composition is administered once daily.

17. The method according to claim 16, wherein said pharmaceutical composition is administered orally in the form of one or two tablets once daily.
